(19) United States
(12) Patent Application Publication
(10) Pub. No.: US 2025/0285273 A1
(43) Pub. Date: Sep. 11, 2025

(54) METHOD OF ANALYZING DERMOSCOPY IMAGES

(71) Applicant: SKLIP INC., Lake Oswego, OR (US)

(72) Inventors: ALEXANDER WITKOWSKI, Wesley Chapel, FL (US); JOANNA LUDZIK, Portland, OR (US)

(21) Appl. No.: 19/074,171

(22) Filed: Mar. 7, 2025

Related U.S. Application Data
(60) Provisional application No. 63/562,279, filed on Mar. 7, 2024.

(51) Int. Cl. G06T 7/00; G16H 30/40

(52) U.S. Cl. CPC G06T 7/0012; G16H 30/40; G06T 2207/30088; G06T 2207/30096; G06T 2207/30168

(57) ABSTRACT

The method of analyzing digital dermoscopy images records, stores and transfers digital dermoscopy images (DDIs) and provides an initial characterization based on clinically accepted dermoscopy criteria. A digital dermoscopy image is received and a determination is made to determine if the digital dermoscopy image meets a quality threshold. If the digital dermoscopy image meets the quality threshold, then a determination is made if the digital dermoscopy image potentially shows a pre-malignant or malignant skin lesion or an atypical melanocytic lesion with uncertain malignant potential. The determination of the potential showing of the pre-malignant or malignant skin lesion or the atypical melanocytic lesion with uncertain malignant potential is performed using an artificial intelligence system or an augmented intelligence system pre-trained with a dataset of diverse dermoscopy images. The results of the determination are visually indicated to the user.

### CROSS-REFERENCE TO RELATED APPLICATIONS

This application claims the benefit of U.S. Provisional Patent Application No. 63/562,279, filed on Mar. 7, 2024, which is hereby incorporated by reference in its entirety.

### BACKGROUND

### Field

The disclosure of the present patent application relates to dermatological evaluations, and particularly to a method of analyzing digital dermoscopy images for providing initial characterizations of features contained therein.

### Description of Related Art

Dermoscopy or dermatoscopy is the examination of the skin using skin surface microscopy (sometimes also referred to as epiluminoscopy or epiluminescent microscopy). Derm (at) oscopy is mainly used to evaluate pigmented skin lesions and makes it easier to diagnose melanoma. Dermoscopy requires a high-quality magnifying lens and a powerful lighting system (a dermatoscope). Computer software is often used to archive dermoscopy images and to allow for expert diagnosis and reporting (i.e., mole mapping). Such diagnoses, however, are performed visually by medical professionals who study the images. This limits the ability of others, such as the patients themselves, to perform initial self-assessments. Thus, a method of analyzing digital dermoscopy images solving the aforementioned problems is desired.

### SUMMARY

The method of analyzing digital dermoscopy images records, stores and transfers digital dermoscopy images (DDIs) and provides an initial characterization based on the clinically accepted dermoscopy three-point checklist (D3PC) criteria and the modified three-point checklist (MD3PC) criteria. The initial characterization from the DDIs aids in the clinical characterization of target skin lesions (TSLs) and/or pigmented skin lesions (PSLs) as suspicious for skin cancers (e.g., melanoma, basal cell carcinoma, or squamous cell carcinoma), or atypical melanocytic nevi with uncertain malignant potential (AMNUMP) (e.g., atypical nevi, spitz nevi, etc.), with ternary output to the user. In use, the DDIs may be collected and recorded using a dermatoscope attached to, or otherwise adapted for use with, the digital camera of a mobile device, such as a smartphone, tablet computer or the like.

A digital dermoscopy image is received by the mobile device and stored in memory thereof. A determination is made to determine if the digital dermoscopy image meets a quality threshold. This determination is made by software running on the mobile device. If the digital dermoscopy image meets the quality threshold, then a determination is made if the digital dermoscopy image potentially shows a pre-malignant or malignant skin lesion or an atypical melanocytic lesion with uncertain malignant potential. This determination is also made by software running on the mobile device. The determination of the potential showing of the pre-malignant or malignant skin lesion (e.g., melanoma, basal cell carcinoma or squamous cell carcinoma) or the atypical melanocytic lesion with uncertain malignant potential (AMNUMP) is performed using an artificial intelligence system or an augmented intelligence system pre-trained with a dataset of diverse dermoscopy images. The results of the determination are visually indicated to the user on the display of the mobile device.

Further, a digital dermoscopy image heatmap overlay is generated, including an overlay for the DDI with different colors representing differing degrees of likelihood of positive dermoscopy features associated with possible pre-malignant and/or malignant tissue (e.g., melanoma, basal cell carcinoma or squamous cell carcinoma) or atypical melanocytic nevi of uncertain malignant potential (AMNUMP). The overlaid heatmap is image is displayed to the user on the display of the mobile device.

The determination of the potential showing of the pre-malignant or malignant skin lesion or the atypical melanocytic nevus of uncertain malignant potential further is performed by feature extraction from the digital dermoscopy image. The features are analyzed by the artificial intelligence system or the augmented intelligence system for the presence of parameters including asymmetry, atypical network, blue-white-grey-violet structures, radial streams, pseudopods, irregular diffuse pigmentation, irregular dots and globules, regression patterns, and combinations thereof. The irregular dots and globules are in the form of round and/or oval structures.

These and other features of the present subject matter will become readily apparent upon further review of the following specification.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A shows a screenshot from a mobile device performing the method of analyzing digital dermoscopy images, particularly showing an initial digital dermoscopy image (DDI) recorded on the mobile device.

FIG. 1B is a screenshot showing an exemplary “wait” screen while the system analyzes the DDI for quality.

FIG. 2A, FIG. 2B, FIG. 2C, FIG. 2D and FIG. 2E show exemplary screenshots used in the method of analyzing digital dermoscopy images in an educational system.

FIG. 3 is a screenshot showing an exemplary error prompt in the educational system.

FIG. 4A, FIG. 4B and FIG. 4C show exemplary subsequent screenshots used in the method of analyzing digital dermoscopy images in an educational system.

FIG. 5A, FIG. 5B, FIG. 5C, FIG. 5D, FIG. 5E and FIG. 5F show exemplary screenshots used in the method of analyzing digital dermoscopy images in a clinical system.

Similar reference characters denote corresponding features consistently throughout the attached drawings.

### DETAILED DESCRIPTION

The method of analyzing digital dermoscopy images records, stores and transfers digital dermoscopy images (DDIs) and provides an initial characterization based on the clinically accepted dermoscopy three-point checklist (D3PC) criteria and the modified three-point checklist (MD3PC) criteria. The initial characterization from the DDIs aids in the clinical characterization of target skin lesions (TSLs) and/or pigmented skin lesions (PSLs) as suspicious for skin cancers (e.g., melanoma, basal cell carcinoma, or squamous cell carcinoma), or atypical melanocytic nevi with uncertain malignant potential (AMNUMP) (e.g., atypical nevi, spitz nevi, etc.), with ternary output to the user. In use, the DDIs may be collected and recorded using a dermatoscope attached to, or otherwise adapted for use with, the digital camera of a mobile device, such as a smartphone, tablet computer or the like. Non-limiting examples of dermatoscope hardware are shown in U.S. Patent Publication Nos. US 2020/0367803 A1 and US 2022/0370005 A1, each of which is hereby incorporated by reference in its entirety.

A digital dermoscopy image is received by the mobile device and stored in memory thereof. A determination is made to determine if the digital dermoscopy image meets a quality threshold. This determination is made by software running on the mobile device. If the digital dermoscopy image meets the quality threshold, then a determination is made if the digital dermoscopy image potentially shows a pre-malignant or malignant skin lesion or an atypical melanocytic lesion with uncertain malignant potential. This determination is also made by software running on the mobile device. The determination of the potential showing of the pre-malignant or malignant skin lesion (e.g., melanoma, basal cell carcinoma or squamous cell carcinoma) or the atypical melanocytic lesion with uncertain malignant potential (AMNUMP) is performed using an artificial intelligence system or an augmented intelligence system pre-trained with a dataset of diverse dermoscopy images. The results of the determination are visually indicated to the user on the display of the mobile device. However, during the initial quality assessment, if the digital dermoscopy image is identified as not being a digital dermoscopy image, or not being of sufficient quality, then the software system operating on the mobile device does not proceed to the next determination step. In this scenario, the output to the user on the display is an error message, such as “ERROR”, for example. If, at the initial determination step, the digital dermoscopy image is confirmed to be a digital dermoscopy image of sufficient quality, then the software proceeds to the second determination step.

A secondary quality test may also be performed before proceeding to the determination of the potential showing of the pre-malignant or malignant skin lesion or the AMNUMP. The digital dermoscopy image may be further evaluated by the software running on the mobile device to determine if the digital dermoscopy image is a) of appropriate technical quality and qualified for the subsequent determination step, or b) not of appropriate technical quality and not qualified for the subsequent determination step. As a non-limiting example, a qualified image may show the imaged target pigmented skin lesion (PSL) is not dry, is not obstructed by hair or other artifacts, and/or is not in a non-qualified anatomical area. If the digital dermoscopy image is not qualified, the software system does not proceed to the subsequent step. In this scenario, the output to the user displays “ERROR” or a similar message. If the digital dermoscopy image is qualified, the software system proceeds to the determination of the potential showing of the pre-malignant or malignant skin lesion or the AMNUMP.

The determination of the potential showing of the pre-malignant or malignant skin lesion or the AMNUMP from the digital dermoscopy image (DDI) is based on a determination if any of the criteria of the D3PC/MD3PC criteria are present in the digital dermoscopy image. If D3PC/MD3PC criteria are not identified (i.e., not present in the DDI), the output to the user on the display of the mobile device may be “NEGATIVE”, as a non-limiting example. For clinical use, a clinical display may be, as a non-limiting example, “UNREMARKABLE”. If at least one D3PC/MD3PC criterion is identified (i.e., present), then the output to the user in an educational system may be, as a non-limiting example, “POSITIVE”, and in a clinical system, may be “SUSPICIOUS”, as a non-limiting example.

It should be understood that any suitable criteria may be used to distinguish the features shown in the DDI. A common method used by licensed healthcare professionals in dermatology, primary care, and other specialties is the dermoscopy three-point checklist (D3PC), as a nonlimiting example, which includes the following: Asymmetry (asymmetry of color and or structure in one or two perpendicular axes); Atypical network (pigment network with irregular holes and thick lines (including round and or oval structures)); and Blue-white structures (any type of blue, white, grey, and or violet color (e.g., a combination of blue-white veil, regression structures, etc.)).

The above may be considered major criteria. Additional nonlimiting minor criteria to consider include: Radial streaming (e.g., streaks, pseudopods, etc.); Irregular diffuse pigmentation (e.g., blotches, dermoscopic islands, etc.); Irregular dots and globules (e.g., round and or oval structures); Any combination of blue, white, grey, and or violet colors suggestive of pigment present in deeper skin layers; and Regression patterns.

Further, a digital dermoscopy image heatmap overlay is generated, including an overlay for the DDI with different colors representing differing degrees of likelihood of positive dermoscopy features associated with possible pre-malignant and/or malignant tissue (e.g., melanoma, basal cell carcinoma or squamous cell carcinoma) or atypical melanocytic nevi of uncertain malignant potential (AMNUMP). The overlaid heatmap is image is displayed to the user on the display of the mobile device.

The determination of the potential showing of the pre-malignant or malignant skin lesion or the atypical melanocytic nevus of uncertain malignant potential further is performed by feature extraction from the digital dermoscopy image. The features are analyzed by the artificial intelligence system or the augmented intelligence system for the presence of parameters including asymmetry, atypical network, blue-white-grey-violet structures, radial streams, pseudopods, irregular diffuse pigmentation, irregular dots and globules, regression patterns, and combinations thereof. The irregular dots and globules are in the form of round and/or oval structures.

The method of analyzing digital dermoscopy images may be applied to an educational system. The educational system may use the method of analyzing digital dermoscopy images to analyze target skin lesions (TSLs) and pigmented skin lesions (PSLs) (e.g., moles) on people not restricted by age. The educational system discussed herein is not intended for clinical triage or diagnosis. The clinical decision regarding triage (e.g., to biopsy, monitor, or other patient management) and/or diagnosis of a TSL and/or PSL of concern should be completed independent of the educational system.

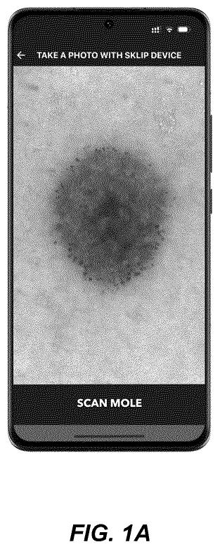

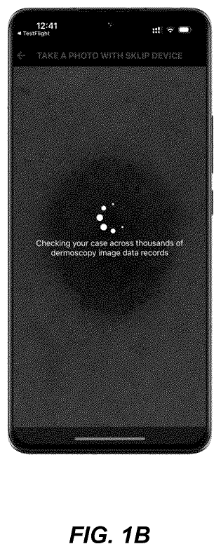

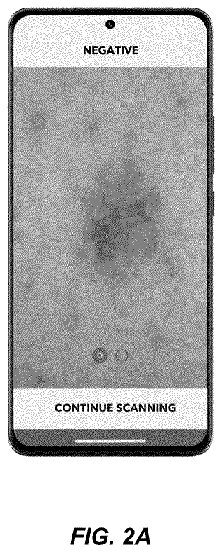

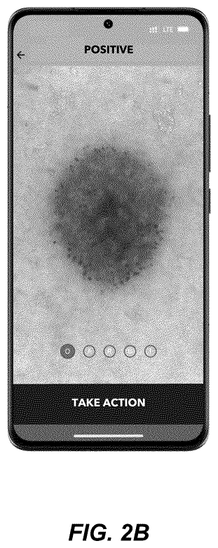

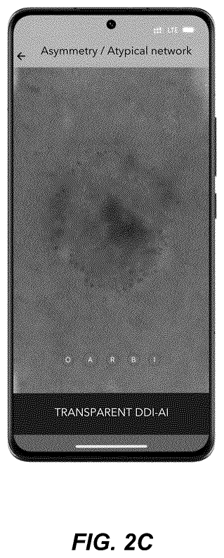

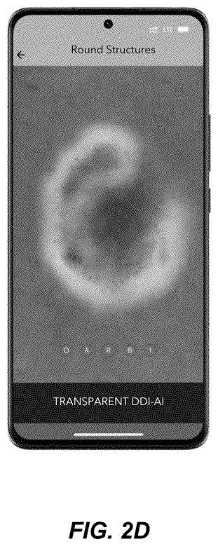

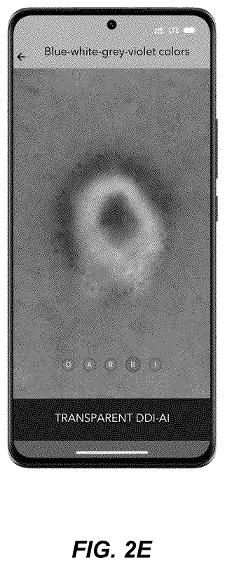

FIG. 1A shows an initial DDI recorded on a mobile device, and FIG. 1B shows an exemplary “wait” screen while the system analyzes the DDI for quality. In FIG. 1A, the user may initiate the determinations may pressing “SCAN MOLE” on the touchscreen display, as a non-limiting example. Assuming that the DDI is of sufficient quality, the educational system provides one of three educational information outputs for each image: “NEGATIVE”; “POSITIVE”; or “ERROR”. “NEGATIVE” indicates limited or no positive D3PC/MD3PC criteria (i.e., no possible immediate concerning dermoscopic features associated with pre-malignancy or malignancy), where one DDI image (the original input DDI) is displayed to the user with information available about the educational information result. FIG. 2A shows an exemplary “NEGATIVE” result.

For the “POSITIVE” output, this indicates positive D3PC/MD3PC criteria (i.e., possible low, moderate, or high concern for pre-malignancy or malignancy). Four DDI graphical digital outputs with information about the educational information result are displayed to the user and can be toggled on the mobile device by swiping the screen left or right, or on a computer by clicking the result window left or right. The first DDI is displayed in its original form with a digital graphic output header text “POSITIVE” (FIG. 2B). The second DDI is displayed with a digital graphic output header text “POSITIVE [Asymmetry/AN]” and a generated digital dermoscopy image heatmap overlay that displays suggested areas within the original input DDI where asymmetry and/or atypical network D3PC/MD3PC umbrella features may be present (FIG. 2C). The third DDI is displayed with a digital graphic output header text “POSITIVE [Round structures],” and a generated digital dermoscopy image heatmap overlay that displays suggested areas within the original input DDI where round and or oval structures D3PC/MD3PC umbrella features may be present (FIG. 2D). The fourth DDI is displayed with a digital graphic output header text “POSITIVE [Blue-white colors or BWGV colors]” and a generated digital dermoscopy image heatmap overlay that displays suggested areas within the original input DDI where blue, white, grey, and or violet color D3PC/MD3PC umbrella features may be present (FIG. 2E). The heatmaps are presented to the user for each individual D3PC/MD3PC feature in three separate images. It should be understood that the term “digital dermoscopy image heatmap,” as used herein, does not refer to a thermogram or thermographic image but rather distinguishes the magnitudes of a particular parameter within the same image by color (similar to how temperature differences and gradients are shown in a thermogram, or how altitudes are shown by color in an altitude map).

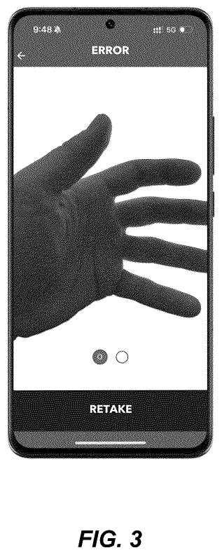

For the “ERROR” output, this indicates that the DDI does not meet the necessary quality criteria or that an assessment of the DDI cannot be made. This result may be returned when the DDI is not of the right type of item (e.g., a DDI taken without dermatoscope hardware) or that the image technical quality is insufficient. The user is then prompted with instructions for how to correctly retake a qualified DDI, as illustrated in FIG. 3.

DDI evaluated with a “POSITIVE” result and having a high likelihood of D3PC/MD3PC positive criteria are marked by the transparent DDI-AI Heatmap overlay with red, orange, and or yellow colors. DDI evaluated by Sklip System Educational with a “POSITIVE” result and have a moderate likelihood of D3PC/MD3PC positive criteria and are marked by the transparent digital dermoscopy image heatmap overlay with teal and/or light blue colors, as non-limiting examples. DDI evaluated with a “POSITIVE” result and having a low likelihood of D3PC/MD3PC positive criteria are marked by the transparent digital dermoscopy image heatmap overlay with dark blue or purple colors, as non-limiting examples.

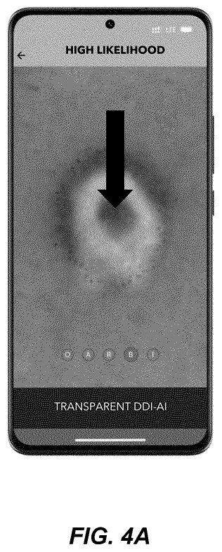

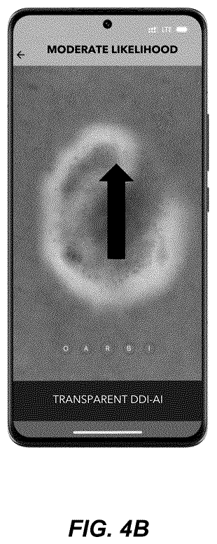

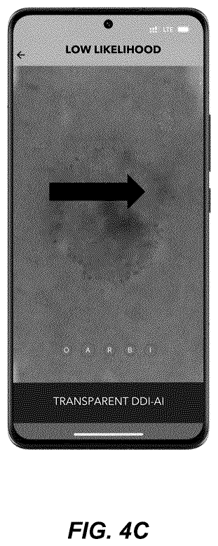

The transparent digital dermoscopy image heatmap overlay provides transparency to the user of what, if any, dermoscopy (dermatoscopy) features are detected by the system. In the non-limiting examples of FIGS. 4A, 4B and 4C, a high likelihood is indicated by a down arrow, a moderate likelihood is indicated by an up arrow, and a low, or absence of, likelihood is indicated by a right arrow.

The method of analyzing digital dermoscopy images may also be used with a clinical system. The clinical system is intended for use by licensed healthcare professionals to assess, triage, biopsy or refer a TSL and/or PSL of concern (TSLC/PSLC) to another licensed healthcare professional for a second opinion. A target TSLC/PSLC may be identified by the patient, their partner, family, other persons, or a licensed healthcare professional. Licensed healthcare professionals may include, but are not limited to, medical doctors (MDs), doctors of osteopathy (DOs), nurse practitioners (NPs), physician assistants and or associates (PAs), dental healthcare providers (e.g., DDSs, DMDs, dental hygienists, etc.) who engage in extraoral skin checks, and/or nurses (e.g., RNs, LPNs, etc.) who engage in skin checks and or biopsies. The clinical system may also be used by laypersons in research settings. Layperson use in clinical settings would also require FDA clearance and/or approval. The clinical system is intended for use on adults over 21 years of age.

The clinical system evaluates DDIs for the presence of at least one D3PC/MD3PC feature, and the user interface graphical output is displayed to the user for triage and/or diagnostic purposes. The clinical system is an adjunctive triage and/or diagnostic standalone device and displays one of three possible outputs to the user regarding the target TSLC/PSLC: “SUSPICIOUS,” “UNREMARKABLE,” or “ERROR”. The error message is displayed if the DDI is not able to be assessed or is unreadable. The clinical system is intended to identify dermoscopic (dermatoscopic) features associated with skin cancers (e.g., melanoma, basal cell carcinoma, and squamous cell carcinoma) and atypical melanocytic nevi of uncertain malignant potential (AMNUMP) (e.g., atypical nevi, spitz nevi, etc.). Triage, diagnosis, and patient management decisions are made by the licensed healthcare professional. PSLCs identified by laypersons should be reported to a licensed healthcare professional when a “SUSPICIOUS” result is received and/or the layperson user does not understand the “UNREMARKABLE” or “ERROR” results.

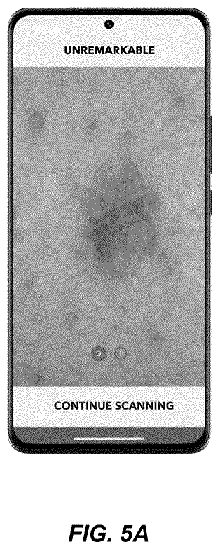

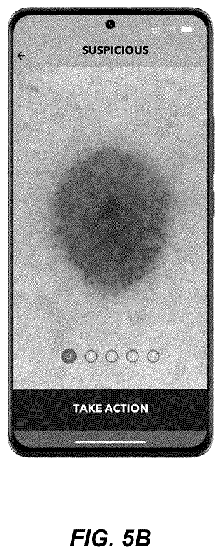

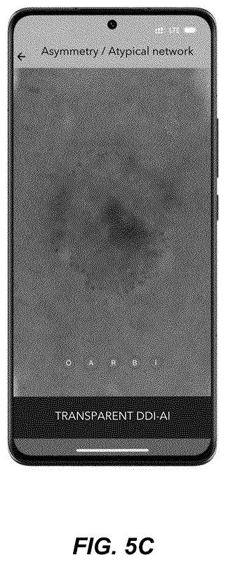

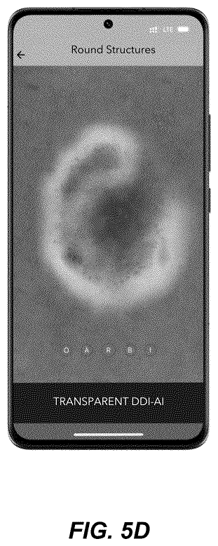

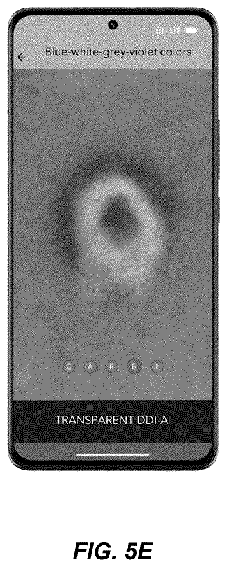

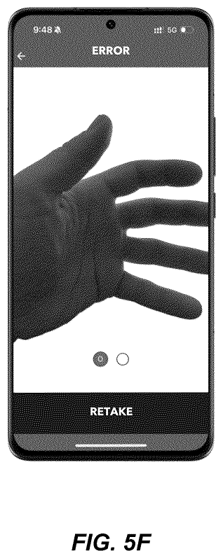

Initially, the user acquires a DDI with appropriate dermoscopic (dermatoscopic) hardware and submits the DDI to the system for evaluation using a similar method as that described above with regard to the educational system. With regard to the “UNREMARKABLE” output, this indicates limited or no positive D3PC/MD3PC features with low concern for pre-malignancy or malignancy. A DDI spot check follow-up within three to six months should be considered. During clinical follow-up, the original DDI may be compared side-by-side with a new DDI of the same PSLC. If there is a change during follow-up by a licensed healthcare professional, then a biopsy should be considered. If there is a change during follow-up by a layperson, then it should be urgently reported to a licensed healthcare professional. One DDI image (the original input DDI) is displayed to the user via graphical output with information available about the clinical result (FIG. 5A).

With regard to a determination and display of “SUSPICIOUS”, this indicates positive D3PC/MD3PC with moderate to high concern for pre-malignancy or malignancy. Additional evaluation is recommended, as determined appropriate by the licensed healthcare professional. This may be a biopsy, or if a biopsy is not taken, then a DDI spot check follow up within three months, or sooner, should be considered. PSLCs identified by laypersons should be reported to a licensed healthcare professional when a “SUSPICIOUS” result is received.

Four DDI are displayed to the user via graphical digital output with information about the clinical result. The first DDI is displayed in its original form with a digital graphic output header with the text “SUSPICIOUS” (FIG. 5B). The second DDI is displayed with a digital graphic output header text “SUSPICIOUS [Asymmetry/AN]” and a generated transparent digital dermoscopy image heatmap overlay that displays suggested areas within the original input DDI where asymmetry and/or atypical network D3PC/MD3PC umbrella features may be present (FIG. 5C). The third DDI is displayed with a digital graphic output header text “SUSPICIOUS [Round structures]” and a generated transparent digital dermoscopy image heatmap overlay that displays suggested areas within the original input DDI where round structures and or oval structure D3PC/MD3PC umbrella features may be present (FIG. 5D). The fourth DDI is displayed with a digital graphic output header text “SUSPICIOUS [Blue-white colors, or BWGV colors]” and a generated transparent digital dermoscopy image heatmap overlay that displays suggested areas within the original input DDI where blue, white, grey, and or violet color D3PC/MD3PC umbrella features may be present (FIG. 5E). The digital dermoscopy image heatmap overlays are presented to the user for each individual D3PC/MD3PC feature in three separate images within the clinical system. It should be understood that the term “heatmap,” as used herein, does not refer to a thermogram or thermographic image but rather distinguishes the magnitudes of a particular parameter within the same image by color (similar to how temperature differences and gradients are shown in a thermogram, or how altitudes are shown by color in an altitude map).

The “ERROR” message is displayed when the DDI does not meet the quality criteria and an assessment of the DDI cannot be made. This result may be returned when the DDI is not of the right sort of item or that the image quality is insufficient. The user is prompted with instructions for how to correctly retake a qualified DDI, as shown in FIG. 5F.

If the clinical system provides an “ERROR” result, the device provides the user with information on how to properly retake a DDI. If the user receives three “ERROR” results in a row for the same target PSLC, the device labelling clearly instructs the user to consult a licensed dermatology healthcare professional. If a licensed healthcare professional receives three “ERROR” results in a row for the same target PSLC, the device labelling clearly instructs the licensed healthcare professional to use their best clinical judgement when choosing PSLC management, independent of the clinical system. If a layperson receives three “ERROR” results in a row for the same target PSLC, the device labelling clearly instructs the layperson to consider reporting their PSLC to a licensed healthcare professional. The use of the clinical system is intended to provide triage and diagnostic clinical assistance. It is not intended to provide a diagnosis that should be made by a licensed pathologist. The clinical system does not provide a specific recommendation for treatment and or management.

The artificial intelligence running on the mobile device was trained by expert dermatologists (dermoscopy experts) who annotated and supervised the algorithm training set using the D3PC/MD3PC to identify dermoscopic features associated with skin cancers (e.g., melanoma, basal cell carcinoma, and squamous cell carcinoma) and atypical melanocytic nevi of uncertain malignant potential (AMNUMP) (e.g., atypical nevi, spitz nevi, etc.). All dermoscopic features of the D3PC/MD3PC are used to interpret DDI that are processed through both the educational and clinical systems. While the use of D3PC versus MD3PC does not influence the output result, or its accuracy, the system's digital graphic outputs prioritize the MD3PC and separates the round and/or oval structures from the atypical network umbrella as an isolated feature for detection and display to the user. This is due to the system's ability to detect round and/or oval structures independent of other dermoscopic criteria with high accuracy. The dermoscopic round and/or oval structures criteria are associated with the highest horizontal growth rate in melanoma skin cancer.

Table 1 below shows the dermoscopic (dermatoscopic) criteria used to train the AI system (showing no difference in evaluation criteria between D3PC and MD3PC) versus the system's user interface (UI) output that includes atypical network in the asymmetry group and places round and or oval structures an independent feature for display to the user.

TABLE 1

| | Modified | User interface (UI) |
|---|---|---|
| Dermoscopy three- | dermoscopy three- | output when DDI is |
| point checklist | point checklist | positive for at least |
| (D3PC) used to | (MD3PC) used | one D3PC/MD3PC |
| evaluate DDI | to evaluate DDI | feature |
| Asymmetry | Asymmetry and/or | Asymmetry and/or |
| (geometric/axial) | atypical network | atypical network |
| Atypical network | Round structures | Round structures |
| (includes round | (including oval | |
| and/or oval | structures) | |
| structures) | | |
| Blue-white structures | Blue-white-grey- | Blue-white-grey-violet |
| (includes grey and | violet colors | colors |
| violet colors) | | |

As discussed above, in addition to the ternary outputs (e.g., “NEGATIVE,” “POSITIVE,” and “ERROR” for the educational system and “UNREMARKABLE,” “SUSPICIOUS,” and “ERROR” for the clinical system), the device produces an additional output when the DDI input is classified as “POSITIVE” (educational) or “SUSPICIOUS” (clinical). This additional output is a generated transparent digital dermoscopy image heatmap overlay showing where possible positive D3PC/MD3PC features are located within the DDI on an X-Y axis, or longitude-latitude axis, where the reference map is the original input DDI. The heatmap images are presented to the user for each individual D3PC/MD3PC feature in three separate images.

DDI with high likelihood of D3PC/MD3PC positive criteria are marked with red, orange, and/or yellow colors. DDI with moderate likelihood of D3PC/MD3PC positive criteria are marked with teal or light blue colors. DDI with low likelihood, or possible absence, of D3PC/MD3PC positive criteria are marked with dark blue or purple colors.

It is to be understood that the method of analyzing digital dermoscopy images is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method of analyzing digital dermoscopy images, comprising:
receiving a digital dermoscopy image;
determining if the digital dermoscopy image meets a quality threshold;
when the digital dermoscopy image meets the quality threshold, determining if the digital dermoscopy image potentially shows a pre-malignant or malignant skin lesion or an atypical melanocytic lesion with uncertain malignant potential, wherein the determination of the potential showing of the pre-malignant or malignant skin lesion or the atypical melanocytic lesion with uncertain malignant potential is performed by an artificial intelligence system or an augmented intelligence system pre-trained with a dataset of diverse dermoscopy images; and
visually indicating to a user results of the determination.

2. The method of analyzing digital dermoscopy images as recited in claim 1, further comprising generating a digital dermoscopy image heatmap overlay, wherein different colors shown in the digital dermoscopy image heatmap overlay represent differing degrees of likelihood of positive dermoscopy features associated with possible pre-malignant and/or malignant tissue or atypical melanocytic nevi of uncertain malignant potential.

3. The method of analyzing digital dermoscopy images as recited in claim 1, wherein the determination of the potential showing of the pre-malignant or malignant skin lesion or the atypical melanocytic nevus of uncertain malignant potential further comprises feature extraction from the digital dermoscopy image, wherein features are analyzed by the artificial intelligence system or the augmented intelligence system for a presence of parameters selected from the group consisting of asymmetry, atypical network, blue-white-grey-violet structures, radial streams, pseudopods, irregular diffuse pigmentation, irregular dots and globules, regression patterns, and combinations thereof.

4. The method of analyzing digital dermoscopy images as recited in claim 3, wherein the irregular dots and globules comprise round and/or oval structures.
